(19) United States
(12) Patent Application Publication
(10) Pub. No.: US 2023/0310290 A1
(43) Pub. Date: Oct. 5, 2023

(54) SUNSCREEN OR DAILY CARE COMPOSITION COMPRISING MICRONIZED TRIS-BIPHENYL TRIAZINE

(71) Applicant: BASF SE, Ludwigshafen am Rhein (DE)

(72) Inventors: Myriam SOHN, Grenzach-Wyhlen (DE); Stanislaw KRUS, Grenzach-Wyhlen (DE); Julie GRUMELARD, Grenzach-Wyhlen (DE)

(21) Appl. No.: 18/024,281

(22) PCT Filed: Sep. 1, 2021
(86) PCT No.: PCT/EP2021/074192

§ 371 Date: Mar. 2, 2023

(30) Foreign Application Priority Data
Sep. 3, 2020 (EP) 20194362.8

(51) Int. Cl. A61K 8/41; A61Q 17/04; A61K 8/49; A61K 8/35; A61K 8/39

(52) U.S. Cl. CPC A61K 8/415; A61Q 17/04; A61K 8/4966; A61K 8/35; A61K 8/39; A61K 2800/412

(57) ABSTRACT

The present invention relates to sunscreen or daily care compositions comprising (i) an ultraviolet absorber water-dispersed composition comprising (a) micronized tris-biphenyl triazine, and (b) decaglyceryl monolaurate; and (ii) at least one oil soluble UVA filter.

The present invention relates to sunscreen or daily care compositions comprising (i) an ultraviolet absorber water-dispersed composition comprising (a) micronized tris-biphenyl triazine, and (b) decaglyceryl monolaurate; and (ii) at least one oil soluble UVA filter, wherein the at least one oil soluble UVA filter is a combination of hexyl 2-[4-(diethylamino)-2-hydroxybenzoyl]benzoate (INCI diethylamino hydroxybenzoyl hexyl benzoate) and 1-(4-(1,1-dimethylethyl)phenyl)-3-(4-methoxyphenyl)propane-1,3-dione (INCI butyl methoxydibenzoylmethane). Furthermore, the present invention relates to the use of an ultraviolet absorber water-dispersed compositions to improve water resistance and/or the sun protection factor of a sunscreen or daily care composition comprising at least one oil soluble UVA filter, wherein the at least one oil soluble UVA filter is a combination of hexyl 2-[4-(diethylamino)-2-hydroxybenzoyl]benzoate (INCI diethylamino hydroxybenzoyl hexyl benzoate) and 1-(4-(1,1-dimethylethyl)phenyl)-3-(4-methoxyphenyl)propane-1,3-dione (INCI butyl methoxydibenzoylmethane).

UV radiation causes harmful effects on the human skin. Beside the acute effect of sunburn of the skin, UV radiation is also known to increase the risk of skin cancer. Furthermore, long time exposure to UV-A and UV-B light can cause phototoxic and photo allergenic reactions on the skin and can accelerate skin aging.

To protect the human skin from UV radiation, various sun protecting UV filters (also referred to as UV absorbers) exist including UV-A filter (also referred to as UVA filter), UV-B filter (also referred to as UVB filter) and broadband filters. These filters are added to sunscreen or daily care compositions. The UV filters are either organic or inorganic, particulate or non-particulate compounds, of which all have a high absorption efficacy in the UV-light range. In general, UV light can be divided into UV-A radiation (320-400 nm) and UV-B radiation (290-320 nm). Depending on the position of the absorption maxima, UV filters are divided into UV-A and UV-B filters. In case an UV filter absorbs both, UV-A and UV-B light, it is referred to as a broadband absorber.

Since 2006, the EU commission has recommended that all sunscreen or daily care compositions should have an UV-A protection factor, which is at least one third of the labelled sun protection factor (SPF), wherein the sun protection factor refers mainly to the UV-B protection.

However, even if main marketed products are providing both UVB and UVA protection according to the current standards, there is a need for improvement of the water resistance. Water resistance is an important performance indicator for end consumers, since users exemplarily need UV protection while swimming or during outdoor activities in summer (sweating), but is yet difficult to be achieved. In addition, a lot of sunscreen or daily care compositions contain UV filter and other ingredients such as dispersing agents, which are frequently under discussion due to some concerns on human safety and skin sensitization potential, especially for a daily use, although they are approved for being used in leave-on compositions.

Against this background, there is an ongoing need for sunscreen or daily care compositions for the daily use, which provide efficient sun protection and water resistance. In particular, it was an object of the present invention to provide a sunscreen or daily care compositions having an improved water resistance and/or an improved sun protection. Further, it was an object of the present invention to provide sunscreen or daily care compositions having reduced components under discussion regarding human safety and skin sensitization concerns. It has been another object of the present invention to provide a sunscreen or daily care composition, which is suitable for daily use. Finally, it was an object of the present invention to enable the use of water-dispersed UV filters to prepare efficient sunscreens with an improved water resistance.

It has surprisingly been found that at least one of these objects can be achieved by the sunscreen or daily care compositions and/or the ultraviolet absorber water-dispersed compositions according to the present invention.

In particular, the inventors of the present application found that the sunscreen or daily care composition according to the present invention provides an efficient UV-A and UV-B protection, thereby also providing an improved water resistance. Furthermore, it has surprisingly been found by the inventors of the present application that the ultraviolet absorber water-dispersed compositions according to the present invention provides improved water resistance and/or improved sun protection factor.

Thus, according to one aspect, the present invention relates to a sunscreen or daily care composition comprising
(i) an ultraviolet absorber water-dispersed composition comprising
(a) micronized 2,4,6-Tri([1,1′-biphenyl]-4-yl)-1,3,5-triazine (INCI tris-biphenyl triazine), and
(b) decaglyceryl monolaurate (INCI Polyglyceryl-10 Laurate); and
(ii) at least one oil soluble UVA filter,
wherein the at least one oil soluble UVA filter is a combination of hexyl 2-[4-(diethylamino)-2-hydroxybenzoyl]benzoate (INCI diethylamino hydroxybenzoyl hexyl benzoate) and 1-(4-(1,1-dimethylethyl)phenyl)-3-(4-methoxyphenyl)propane-1,3-dione (INCI butyl methoxydibenzoylmethane).

In the following, preferred embodiments of the above sunscreen or daily care composition are described in further detail. It is to be understood that each preferred embodiment is relevant on its own as well as in combination with other preferred embodiments.

In a preferred embodiment A1 of the first aspect, the sunscreen or daily care composition further comprises at least one additional UV filter.

In a preferred embodiment A2 of the first aspect, the at least one additional UV filter is selected from the group consisting of water soluble UVA filters, UVB filters and/or broadband filters, preferably wherein the sunscreen or daily care composition comprises disodium phenyl dibenzimidazole tetrasulfonate as water soluble UVA filter, a UVB filter selected from the group consisting of 4,4′,4″-(1,3,5-triazine-2,4,6-triyltriimino)tris-benzoic acid-tris(2-ethylhexyl)ester (INCI ethylhexyl triazone), 4,4′-[[6-[[4-[[(1,1-dimethylethyl)amino]carbonyl]phenyl]amino]-1,3,5-triazin-2,4-diyl]diimino]bis-benzoic acid-bis(2-ethylhexyl)ester (INCI Diethylhexyl Butamido Triazone), phenylbenzimidazole sulfonic acid, and combinations thereof and/or a broadband filter selected from the group consisting of bis-ethylhexyloxyphenol methoxyphenyl triazine, 2-(2H-benzotriazol-2-yl)-6-[(2-ethylhexyloxy)methyl]-4-methylphenol, 2-(2H-benzotriazol-2-yl)-4-methyl-6-[2-methyl-3-[1,3,3,3-tetramethyl-1-[(trimethylsilyl)oxy]-1-disiloxanyl]propyl]phenol (INCI drometrizole trisiloxane), and combinations thereof.

In a preferred embodiment A3 of the first aspect, the sunscreen or daily care composition comprises
(i) from 0.01 to 10 wt.-%, preferably from 0.1 to 8 wt.-%, of micronized 2,4,6-Tri([1,1′-biphenyl]-4-yl)-1,3,5-triazine (INCI tris-biphenyl triazine), and
   from 0.01 to 8 wt.-%, preferably from 0.1 to 5 wt.-%, of decaglyceryl monolaurate (INCI Polyglyceryl-10 Laurate); and
(ii) from 0.01 to 20 wt.-%, preferably from 2 to 15 wt.-%, of the at least one oil soluble UVA filter,
each based on the total weight of the sunscreen or daily care composition.
   In a preferred embodiment A4 of the first aspect, the sunscreen or daily care composition comprises
(i) from 0.01 to 10 wt.-%, preferably from 0.1 to 8 wt.-%, of micronized 2,4,6-Tri([1,1′-biphenyl]-4-yl)-1,3,5-triazine (INCI tris-biphenyl triazine), based on the total weight of the sunscreen or daily care composition, and
wherein the weight ratio of decaglyceryl monolaurate (INCI Polyglyceryl-10 Laurate) to micronized 2,4,6-Tri([1,1′-biphenyl]-4-yl)-1,3,5-triazine (INCI tris-biphenyl triazine) is from 0.05 to 0.8, preferably from 0.1 to 0.5, and
(ii) from 0.01 to 10 wt.-%, preferably from 1 to 10 wt.-%, of diethylamino hydroxybenzoyl hexyl benzoate, based on the total weight of the sunscreen or daily care composition, and from 0.01 to 5 wt.-%, preferably from 1 to 5 wt.-%, of butyl methoxydibenzoylmethane, based on the total weight of the sunscreen or daily care composition.

In a preferred embodiment A5 of the first aspect, the sunscreen or daily care composition does not comprise Decyl b-D-glucoside (INCI decyl glucoside), 2-hydroxy-4-methoxybenzophenone (INCI oxybenzone), ethylhexyl-2-cyano-3,3-diphenyl-acrylate (INCI octocrylene), 2-ethylhexyl-(2E)-3-(4-methoxyphenyl)acrylate (INCI ethylhexyl methoxy cinnamate), and/or isoamyl-4-methoxycinnamate (INCI isoamyl-p-methoxy cinnamate).

In a second aspect, the present invention relates to the use of an ultraviolet absorber water-dispersed composition comprising
(a) micronized 2,4,6-Tri([1,1′-biphenyl]-4-yl)-1,3,5-triazine (INCI tris-biphenyl triazine), and
(b) at least one polyglycerol monoalkyl ester
   to improve water resistance and/or the sun protection factor of a sunscreen or daily care composition comprising at least one oil soluble UVA filter, wherein the at least one oil soluble UVA filter is a combination of hexyl 2-[4-(diethylamino)-2-hydroxybenzoyl]benzoate (INCI diethylamino hydroxybenzoyl hexyl benzoate) and 1-(4-(1,1-dimethylethyl)phenyl)-3-(4-methoxyphenyl)propane-1,3-dione (INCI butyl methoxydibenzoylmethane).

In a preferred embodiment B1 of the second aspect, the at least one polyglycerol monoalkyl ester is selected from the group consisting of decaglyceryl caprate, decaglyceryl monolaurate, decaglyceryl myristate, decaglyceryl oleate, decaglyceryl stearate, decaglyceryl isostearate, hexaglyceryl cap rate, hexaglyceryl laurate, hexaglyceryl myristate, hexaglyceryl oleate, hexaglyceryl stearate, hexaglyceryl isostearate, pentaglyceryl caprate, pentaglyceryl laurate, pentaglyceryl myristate, pentaglyceryl oleate, pentaglyceryl stearate, pentaglyceryl isostearate, and combinations thereof, preferably wherein the at least one polyglycerol monoalkyl ester is decaglyceryl monolaurate (INCI Polyglyceryl-10 Laurate).

In a preferred embodiment B2 of the second aspect, the ultraviolet absorber water-dispersed composition comprises from 10 to 70 wt.-%, preferably from 40 to 60 wt.-%, of micronized 2,4,6-Tri([1,1′-biphenyl]-4-yl)-1,3,5-triazine (INCI tris-biphenyl triazine), and/or from 1 to 25 wt.-%, preferably from 2 to 20 wt.-%, of the at least one polyglycerol monoalkyl ester, each based on the total weight of the ultraviolet absorber water-dispersed composition.

In a preferred embodiment B3 of the second aspect, the sunscreen or daily care composition comprises from 0.01 to 20 wt.-%, preferably from 2 to 15 wt.-%, of the at least one oil soluble UVA filter, preferably wherein the at least one oil soluble UVA filter is selected from the group consisting of hexyl 2-[4-(diethylamino)-2-hydroxybenzoyl]benzoate (INCI diethylamino hydroxybenzoyl hexyl benzoate), 1-(4-(1,1-dimethylethyl)phenyl)-3-(4-methoxyphenyl)propane-1,3-dione (INCI butyl methoxydibenzoylmethane), and combinations thereof, based on the total weight of the sunscreen or daily care composition.

In a preferred embodiment B4 of the second aspect, the sunscreen or daily care composition does not comprise Decyl b-D-glucoside (INCI decyl glucoside), 2-hydroxy-4-methoxybenzophenone (INCI oxybenzone), ethylhexyl-2-cyano-3,3-diphenyl-acrylate (INCI octocrylene), 2-ethylhexyl-(2E)-3-(4-methoxyphenyl)acrylate (INCI ethylhexyl methoxy cinnamate), and/or isoamyl-4-methoxycinnamate (INCI isoamyl-p-methoxy cinnamate).

In a preferred embodiment B5 of the second aspect, the sunscreen or daily care composition further comprises at least one additional UV filter, preferably wherein the at least one additional UV filter is selected from the group consisting of water soluble UVA filters, UVB filters and/or broadband filters.

In a third aspect, the present invention relates to the sunscreen or daily care composition as defined herein for use in the administration to sensitive skin.

### DETAILED DESCRIPTION

Before describing in detail exemplary embodiments of the present invention, definitions which are important for understanding the present invention are given.

As used in this specification and in the appended claims, the singular forms of “a” and “an” also include the respective plurals unless the context clearly dictates otherwise. In the context of the present invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably ±10%, even more preferably ±5%, and in particular ±2%. It is to be understood that the term “comprising” is not limiting. For the purposes of the present invention the term “consisting of” is considered to be a preferred embodiment of the term “comprising of”. If hereinafter a group is defined to comprise at least a certain number of embodiments, this is meant to also encompass a group which preferably consists of these embodiments only. Furthermore, the terms “first”, “second”, “third” or “(a)”, “(b)”, “(c)”, “(d)” etc. and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. In case the terms “first”, “second”, “third” or “(a)”, “(b)”, “(c)”, “(d)”, “i”, “ii” etc. relate to steps of a method or use or assay there is no time or time interval coherence between the steps, i.e. the steps may be carried out simultaneously or there may be time intervals of seconds, minutes, hours, days, weeks, months or even years between such steps, unless otherwise indicated in the application as set forth herein above or below. It is to be understood that this invention is not limited to the particular methodology, protocols, reagents etc. described herein as these may vary. It is also to be understood that the terminology used herein is for the purpose of describing particular embodiments only, and is not intended to limit the scope of the present invention that will be limited only by the appended claims. Unless defined otherwise, all technical and scientific terms used herein have the same meanings as commonly understood by one of ordinary skill in the art.

As used herein the term “does not comprise” or “free of” means in the context that the composition of the present invention is free of a specific compound or group of compounds, which may be combined under a collective term, that the composition does not comprise said compound or group of compounds in an amount of more than 0.8% by weight, based on the total weight of the composition. Furthermore, it is preferred that the composition according to the present invention does not comprise said compounds or group of compounds in an amount of more than 0.5% by weight, preferably the composition does not comprise said compounds or group of compounds at all.

When referring to compositions and the weight percent of the therein comprised ingredients it is to be understood that according to the present invention the overall amount of ingredients does not exceed 100% (±1% due to rounding).

The term “sunscreen composition” or “sunscreen” refers to any topical product, which absorbs and which may further reflect and scatter certain parts of UV radiation. Thus, the term “sunscreen composition” is to be understood as not only including sunscreen compositions, but also any cosmetic compositions that provide UV protection. The term “topical product” refers to a product that is applied to the skin and can refer, e.g., to sprays, lotions, creams, oils, foams, powders, or gels. According to the present invention the sunscreen composition may comprise one or more active agents, e.g., organic and inorganic UV filters, as well as other ingredients or additives, e.g., emulsifiers, emollients, viscosity regulators, stabilizers, preservatives, or fragrances.

The term “daily care composition” refers to any topical product, which absorbs and which may further reflect and scatter certain parts of UV radiation and is used as an everyday care product for the human body, e.g. for face or body. The daily care composition may comprise one or more active agents, e.g., organic or inorganic UV filters, as well as other ingredients or additives, e.g., emulsifiers, emollients, viscosity regulators, stabilizers, preservatives, or fragrances. Suitable daily care composition are according to the present invention, e.g. leave-on products for face and body, e.g. sunscreen compositions, decorative preparations, and skin care preparations.

Suitable decorative preparations are, e.g., lipsticks, nail varnishes, eye shadows, mascaras, dry and moist make-up, rouge, powders, depilatory agents and suntan lotions.

Suitable skin care preparations are e.g., moisturizing, refining, and lifting preparations.

The cited daily care compositions can be in the form of creams, ointments, pastes, foams, gels, lotions, powders, make-ups, sprays, sticks or aerosols.

The term “UV filter” or “ultraviolet filter” as used herein refers to organic or inorganic compounds, which can absorb and may further reflect and scatter UV radiation caused by sunlight. UV-filter can be classified based on their UV protection curve as UV-A, UV-B, or broadband filters.

Water soluble UV filters have a solubility in water of at least 2% by weight, preferably at least 3% by weight, more preferably at least 5% by weight.

Oil soluble UV filters have a solubility in common cosmetic oils, such as C₁₂-C₁₅-alkyl benzoate, dibutyl adipate, diisopropyl sebacate, phenethyl benzoate, or dicaprylyl carbonate of at least 2% by weight, preferably at least 5% by weight, more preferably at least 7% by weight.

The term “emollient” relates to cosmetic specific oils used for protecting, moisturizing and lubricating the skin. The word emollient is derived from the Latin word mollire, to soften. In general, emollients prevent evaporation of water from the skin by forming an occlusive coating. They can be divided into different groups depending on their polarity index.

The term “polarity index” refers to non-polar or polar oils. Non-polar oils are mainly based on hydrocarbons and lack an electronegative element, such as oxygen. In contrast, polar oils contain heteroatoms that differ in electronegativity, which results in a dipole moment. However, such oils are still insoluble in water, i.e. hydrophobic. The polarity index can be determined by measuring the interfacial tension between the respective oil and water.

The term “C₁₂-C₁₅ alkyl benzoate” refers to esters of benzoic acid with fatty alcohols containing a C₁₂-C₁₅-alkyl chain. C₁₂-C₁₅ alkyl chain is defined as an alkyl chain with C₁₂, C₁₃, C₁₄ or C₁₅ chain length.

The term “sensitive skin” refers to skin of which the natural barrier function is weakened and has broken due to a trigger. A trigger can be for example cold weather, extremely hot water and critical ingredients, which may be included in sunscreen or daily care compositions.

The term “sun protection factor (SPF)” as used herein indicates how well the skin is protected by a sunscreen composition mainly from UV-B radiation. In particular, the factor indicates how much longer the protected skin may be exposed to the sun without getting a sunburn in comparison to untreated skin. For example, if a sunscreen composition with an SPF of 15 is evenly applied to the skin of a person usually getting a sunburn after 10 minutes in the sun, the sunscreen allows the skilled person to stay in the sun 15 times longer. In other words, SPF 15 means that 1/15 of the burning UV radiation will reach the skin, assuming sunscreen is applied evenly at a thick dosage of 2 milligrams per square centimeter (mg/cm²).

The definition of “broadband” protection (also referred to as broad-spectrum or broad protection) is based on the “critical wavelength”. For broadband coverage, UV-B and UV-A protection must be provided. According to the US requirements, a critical wavelength of at least 370 nm is required for achieving broad spectrum protection. Furthermore, it is recommended by the European Commission that all sunscreen or cosmetic compositions should have an UV-A protection factor, which is at least one third of the labelled sun protection factor (SPF), e.g. if the sunscreen composition has an SPF of 30 the UVA protection factor has to be at least 10.

The term “critical wavelength” is defined as the wavelength at which the area under the UV protection curve (% protection versus wavelength) represents 90% of the total area under the curve in the UV region (290-400 nm). For example, a critical wavelength of 370 nm indicates that the protection of the sunscreen composition is not limited to the wavelengths of UV-B, i.e. wavelengths from 290-320 nm, but extends to 370 nm in such a way that 90% of the total area under the protective curve in the UV region are reached at 370 nm.

The term “administration” refers to the application of a sunscreen or daily care composition to the skin of a person.

Preferred embodiment regarding the sunscreen or daily care composition as well as the use thereof to protect skin against UV radiations and the use of the ultraviolet absorber water-dispersed composition to improve the water resistance and/or the sun protection factor are described hereinafter. It is to be understood that the preferred embodiments of the invention are preferred alone or in combination with each other.

As indicated above, the present invention relates in one embodiment to a sunscreen or daily care composition comprising
(i) an ultraviolet absorber water-dispersed composition comprising
(a) micronized 2,4,6-Tri([1,1′-biphenyl]-4-yl)-1,3,5-triazine (INCI tris-biphenyl triazine), and
(b) decaglyceryl monolaurate (INCI Polyglyceryl-10 Laurate); and
(ii) at least one oil soluble UVA filter,
wherein the at least one oil soluble UVA filter is a combination of hexyl 2-[4-(diethylamino)-2-hydroxybenzoyl]benzoate (INCI diethylamino hydroxybenzoyl hexyl benzoate) and 1-(4-(1,1-dimethylethyl)phenyl)-3-(4-methoxyphenyl)propane-1,3-dione (INCI butyl methoxydibenzoylmethane).

In connection with the present invention, the following preferred embodiments regarding the sunscreen or daily care composition are relevant.

Any known process suitable for the preparation of microparticles can be used for the preparation of the micronized tris-biphenyl triazine. The sparingly soluble organic compound tris-biphenyl triazine which is used in the present invention is present in the micronized state and is preferably prepared by wet-milling processes.

As milling apparatus for the preparation of the micronized tris-biphenyl triazine there may be used, for example, a jet mill, ball mill, vibratory mill or hammer mill, preferably a high-speed mixing mill.

Tris-biphenyl triazine is micronized according to the present invention preferably in a ball mill to the desired mean particle size in the presence of water and of Polyglyceryl-10 Laurate which acts as dispersing agent. Other excipients may be added to the preparation, such as thickeners, anti-foaming agents, pH-adjusters, preservatives. Moreover, such micronized tris-biphenyl triazine formulations do not agglomerate and they remain in a dispersed form and do not readily settle.

The mean particle diameter of micronized tris-biphenyl triazine after dispersion process is preferably from 10-2000 nm, more preferably from 20-1500 nm, and further preferably ranging from 50-1000 nm. In the case of less than 10 nm, absorption in the UV-A region may be reduced, and in the case of more than 2000 nm, the ultraviolet prevention effect may be reduced and agglomeration and precipitation may be developed over time. It should be noted that, in this specification, a mean particle diameter means a mean particle diameter.

Examples of other excipients which may be used for the preparation of micronized tris-biphenyl triazine are rheology modifiers, solvents, pH adjuster, antifoaming agents, and preservatives.

Rheology modifiers are optionally added to the UV protection composition which help to stabilize across the time such composition. Examples for aqueous thickeners are represented by natural ingredients and their derivatives such as gums and alginates or by synthetic/semi-synthetic ingredients such as modified starch, modified cellulose and polyacrylates. A preferred rheology modifier is xanthan gum.

Suitable solvents for the grinding process are water, brine, (poly-)ethylenglycol, glycerine or cosmetically acceptable oils. Other suitable solvents are disclosed in the IPCOM No 000031257D in the chapters “esters of fatty acids”, “natural and synthetic triglycerides including glyceryl esters and derivatives”, “pearlescent waxes”, “hydrocarbon oils”, and “silicones or siloxanes”. Preferred solvents are water, butylene glycol, and caprylyl glycol.

Suitable pH adjusters are NaOH, TEA, and citric acid.

A suitable antifoaming agent is simethicone.

A suitable preservatives is COSING Annex V.

Preferably, the ultraviolet absorber water-dispersed composition comprises decaglyceryl monolaurate from 3 to 25 wt.-%, more preferably from 5 to 20 wt.-%, and in particular from 10 to 14 wt.-%, based on the total amount of the ultraviolet absorber water-dispersed composition.

Preferably, the ultraviolet absorber water-dispersed composition comprises the micronized 2,4,6-Tri([1,1′-biphenyl]-4-yl)-1,3,5-triazine (INCI tris-biphenyl triazine) from 30 to 70 wt.-%, more preferably from 40 to 60 wt.-%, and in particular from 45 to 55 wt.-%, based on the total amount of the ultraviolet absorber water-dispersed composition.

According to the present invention, the at least one oil soluble UVA filter is a combination of hexyl 2-[4-(diethylamino)-2-hydroxybenzoyl]benzoate (INCI diethylamino hydroxybenzoyl hexyl benzoate) and 1-(4-(1,1-dimethylethyl)phenyl)-3-(4-methoxyphenyl)propane-1,3-dione (INCI butyl methoxydibenzoylmethane).

In a preferred embodiment, the at least one oil soluble UVA filter comprises hexyl 2-[4-(diethylamino)-2-hydroxybenzoyl]benzoate (INCI diethylamino hydroxybenzoyl hexyl benzoate) and 1-(4-(1,1-dimethylethyl)phenyl)-3-(4-methoxyphenyl)propane-1,3-dione (INCI butyl methoxydibenzoylmethane) in a weight ratio of 10:1 to 1:5, preferably of 5:1 to 1:2, more preferably of 3:1 to 1:1, and in particular of about 2:1.

In one embodiment of the present invention, the sunscreen or daily care composition further comprises at least one additional UV filter.

In this connection it is to be understood that the at least one additional UV filter is different to micronized tris-biphenyl triazine and different to the at least one oil soluble UVA filter. The at least one additional UV filter may exemplarily be a water soluble UVA filter, an UVB filter, and/or a broadband filter.

In one embodiment of the present invention, the at least one additional UV filter is selected from the group consisting of water soluble UVA filters, UVB filters and/or broadband filters. In this connection, the water soluble UVA filter is preferably disodium phenyl dibenzimidazole tetrasulfonate, the UVB filter is preferably selected from the group consisting of 4,4′,4″-(1,3,5-triazine-2,4,6-triyltriimino)tris-benzoic acid-tris(2-ethylhexyl)ester (INCI ethylhexyl triazone), 4,4′-[[6-[[4-[[(1,1-dimethylethyl)amino]carbonyl]phenyl]amino]-1,3,5-triazin-2,4-diyl]diimino]bis-benzoic acid-bis(2-ethylhexyl)ester (INCI Diethylhexyl Butamido Triazone), and phenylbenzimidazole sulfonic acid and the broadband filter is preferably selected from the group consisting of 2-(2H-benzotriazol-2-yl)-6-[(2-ethylhexyloxy)methyl]-4-methylphenol, bis-ethylhexyloxyphenol methoxyphenyl triazine, 2-(2H-benzotriazol-2-yl)-4-methyl-6-[2-methyl-3-[1,3,3,3-tetramethyl-1-[(trimethylsilyl)oxy]-1-disiloxanyl]propyl]phenol (INCI drometrizole trisiloxane), and combinations thereof.

In a preferred embodiment, the sunscreen or daily care composition comprises disodium phenyl dibenzimidazole tetrasulfonate as water soluble UVA filter.

In another preferred embodiment, the sunscreen or daily care composition comprises at least one UVB filter selected from the group consisting of 4,4′,4″-(1,3,5-triazine-2,4,6-triyltriimino)tris-benzoic acid-tris(2-ethylhexyl)ester (INCI ethylhexyl triazone), 4,4′-[[6-[[4-[[(1,1-dimethylethyl)amino]carbonyl]phenyl]amino]-1,3,5-triazin-2,4-diyl]diimino]bis-benzoic acid-bis(2-ethylhexyl)ester (INCI Diethylhexyl Butamido Triazone), phenylbenzimidazole sulfonic acid, and combinations thereof. In a particular embodiment, the sunscreen or daily care composition comprises 4,4′,4″-(1,3,5-triazine-2,4,6-triyltriimino)tris-benzoic acid-tris(2-ethylhexyl)ester (INCI ethylhexyl triazone) as UVB filter.

In another preferred embodiment, the sunscreen or daily care composition comprises at least one broadband filter selected from the group consisting of 2-(2H-benzotriazol-2-yl)-6-[(2-ethylhexyloxy)methyl]-4-methylphenol, bis-ethylhexyloxyphenol methoxyphenyl triazine, 2-(2H-benzotriazol-2-yl)-4-methyl-6-[2-methyl-3-[1,3,3,3-tetramethyl-1-[(trimethylsilyl)oxy]-1-disiloxanyl]propyl]phenol (INCI drometrizole trisiloxane), and combinations thereof. In a particular embodiment, the sunscreen or daily care composition comprises bis-ethylhexyloxyphenol methoxyphenyl triazine as broadband filter.

In yet another preferred embodiment, the sunscreen or daily care composition comprises at least one additional UV filter selected from the group consisting of disodium phenyl dibenzimidazole tetrasulfonate, 4,4′,4″-(1,3,5-triazine-2,4,6-triyltriimino)tris-benzoic acid-tris(2-ethylhexyl)ester (INCI ethylhexyl triazone), 4,4′-[[6-[[4-[[(1,1-dimethylethyl)amino]carbonyl]phenyl]amino]-1,3,5-triazin-2,4-diyl]diimino]bis-benzoic acid-bis(2-ethylhexyl)ester (INCI Diethylhexyl Butamido Triazone), phenylbenzimidazole sulfonic acid, 2-(2H-benzotriazol-2-yl)-6-[(2-ethylhexyloxy)methyl]-4-methylphenol, bis-ethylhexyloxyphenol methoxyphenyl triazine, 2-(2H-benzotriazol-2-yl)-4-methyl-6-[2-methyl-3-[1,3,3,3-tetramethyl-1-[(trimethylsilyl)oxy]-1-disiloxanyl]propyl]phenol (INCI drometrizole trisiloxane), and combinations thereof.

In a particular embodiment, the sunscreen or daily care composition 4,4′,4″-(1,3,5-triazine-2,4,6-triyltriimino)tris-benzoic acid-tris(2-ethylhexyl)ester (INCI ethylhexyl triazone) and/or bis-ethylhexyloxyphenol methoxyphenyl triazine as at least one additional UV filter.

In a particular embodiment, the sunscreen or daily care composition comprises 4,4′-[[6-[[4-[[(1,1-dimethylethyl)amino]carbonyl]phenyl]amino]-1,3,5-triazin-2,4-diyl]diimino]bis-benzoic acid-bis(2-ethylhexyl)ester (INCI Diethylhexyl Butamido Triazone) and/or bis-ethylhexyloxyphenol methoxyphenyl triazine as at least one additional UV filter.

The ultraviolet absorber water-dispersed composition is preferably comprised in the sunscreen or daily care composition from 0.1 to 30 wt.-%, more preferably from 0.5 to 25 wt.-%, even more preferably from 1 to 20 wt.-%, and in particular from 2 to 16 wt.-%, based on the total weight of the sunscreen or daily care composition.

Micronized tris-biphenyl triazine is preferably comprised in the sunscreen or daily care composition from 0.01 to 10 wt.-%, more preferably from 0.1 to 8 wt.-%, and in particular from 0.5 to 8 wt.-%, based on the total weight of the sunscreen or daily care composition.

Decaglyceryl monolaurate is preferably comprised in the sunscreen or daily care composition from 0.01 to 10 wt.-%, more preferably from 0.01 to 8 wt.-%, and in particular from 0.1 to 5 wt.-%, based on the total weight of the sunscreen or daily care composition.

The weight ratio of decaglyceryl monolaurate to micronized tris-biphenyl triazine is preferably from 0.05 to 0.8 and more preferably from 0.1 to 0.5.

The at least one oil soluble UVA filter is preferably comprised in the sunscreen or daily care composition from 0.01 to 25 wt.-%, more preferably from 0.1 to 20 wt.-%, and in particular from 2 to 15 wt.-%, based on the total weight of the sunscreen or daily care composition.

In one embodiment of the present invention, the sunscreen or daily care composition comprises
(i) from 0.01 to 10 wt.-%, preferably from 0.1 to 8 wt.-%, of micronized tris-biphenyl triazine, and
   from 0.01 to 8 wt.-%, preferably from 0.1 to 5 wt.-%, of decaglyceryl monolaurate; and
(ii) from 0.01 to 20 wt.-%, preferably from 2 to 15 wt.-%, of the at least one oil soluble UVA filter,
each based on the total weight of the sunscreen or daily care composition.
   In a preferred embodiment, the sunscreen or daily care composition comprises
(i) from 0.01 to 10 wt.-%, preferably from 0.1 to 8 wt.-%, of micronized tris-biphenyl triazine, and
   from 0.01 to 8 wt.-%, preferably from 0.1 to 5 wt.-%, of decaglyceryl monolaurate; and
(ii) from 0.01 to 10 wt.-%, preferably from 1 to 10 wt.-%, of diethylamino hydroxybenzoyl hexyl benzoate, and from 0.01 to 5 wt.-%, preferably from 1 to 5 wt.-%, of butyl methoxydibenzoylmethane;
each based on the total weight of the sunscreen or daily care composition.

In a preferred embodiment, sunscreen or daily care composition comprises
(i) from 0.01 to 10 wt.-%, preferably from 0.1 to 8 wt.-%, of micronized 2,4,6-Tri([1,1′-biphenyl]-4-yl)-1,3,5-triazine (INCI tris-biphenyl triazine), based on the total weight of the sunscreen or daily care composition, and

wherein the weight ratio of decaglyceryl monolaurate (INCI Polyglyceryl-10 Laurate) to micronized 2,4,6-Tri([1,1′-biphenyl]-4-yl)-1,3,5-triazine (INCI tris-biphenyl triazine) is from 0.05 to 0.8, preferably from 0.1 to 0.5, and
(ii) from 0.01 to 10 wt.-%, preferably from 1 to 10 wt.-%, of diethylamino hydroxybenzoyl hexyl benzoate, based on the total weight of the sunscreen or daily care composition, and from 0.01 to 5 wt.-%, preferably from 1 to 5 wt.-%, of butyl methoxydibenzoylmethane, based on the total weight of the sunscreen or daily care composition.

In a further preferred embodiment of the present invention, the sunscreen or daily care composition comprises
(i) from 0.01 to 10 wt.-%, preferably from 0.1 to 8 wt.-%, of micronized tris-biphenyl triazine, and
   from 0.01 to 8 wt.-%, preferably from 0.1 to 5 wt.-%, of decaglyceryl monolaurate;
(ii) from 0.01 to 20 wt.-%, preferably from 2 to 15 wt.-%, of the at least one oil soluble UVA filter; and
(iii) from 0.01 to 20 wt.-%, preferably from 1 to 15 wt.-%, of at least one additional UV filter, preferably selected from the group consisting of water soluble UVA filters, UVB filters and/or broadband filters,
each based on the total weight of the sunscreen or daily care composition.

In another preferred embodiment of the present invention, the sunscreen or daily care composition comprises
(i) from 0.01 to 10 wt.-%, preferably from 0.1 to 8 wt.-%, of micronized tris-biphenyl triazine, and
   from 0.01 to 8 wt.-%, preferably from 0.1 to 5 wt.-%, of decaglyceryl monolaurate; and
(ii) from 0.01 to 10 wt.-%, preferably from 1 to 10 wt.-%, of diethylamino hydroxybenzoyl hexyl benzoate, and from 0.01 to 5 wt.-%, preferably from 1 to 5 wt.-%, of butyl methoxydibenzoylmethane, and
(iii) from 0.01 to 15 wt.-%, preferably from 1 to 10 wt.-%, of at least one additional UV filter selected from the group consisting of 4,4′,4″-(1,3,5-triazine-2,4,6-triyltriimino)tris-benzoic acid-tris(2-ethylhexyl)ester (INCI ethylhexyl triazone), bis-ethylhexyloxyphenol methoxyphenyl triazine, and combinations thereof,
each based on the total weight of the sunscreen or daily care composition.

In another preferred embodiment of the present invention, the sunscreen or daily care composition comprises
(i) from 0.01 to 10 wt.-%, preferably from 0.1 to 8 wt.-%, of micronized tris-biphenyl triazine, and
   from 0.01 to 8 wt.-%, preferably from 0.1 to 5 wt.-%, of decaglyceryl monolaurate; and
(ii) from 0.01 to 10 wt.-%, preferably from 1 to 10 wt.-%, of diethylamino hydroxybenzoyl hexyl benzoate, and from 0.01 to 5 wt.-%, preferably from 1 to 5 wt.-%, of butyl methoxydibenzoylmethane, and
(iii) from 0.01 to 15 wt.-%, preferably from 1 to 10 wt.-%, of at least one additional UV filter selected from the group consisting of 4,4′-[[6-[[4-[[(1,1-dimethylethyl)amino]carbonyl]phenyl]amino]-1,3,5-triazin-2,4-diyl]diimino]bis-benzoic acid-bis(2-ethylhexyl)ester (INCI Diethylhexyl Butamido Triazone), bis-ethylhexyloxyphenol methoxyphenyl triazine, and combinations thereof,
each based on the total weight of the sunscreen or daily care composition.

As indicated above, the sunscreen or daily care composition is preferably free of certain critical compound being under discussion in view of health and skin sensitization concern.

Decyl glucoside may be named as one example for a critical ingredient. 2-hydroxy-4-methoxybenzophenone (INCI oxybenzone), ethylhexyl-2-cyano-3,3-diphenyl-acrylate (INCI octocrylene), 2-ethylhexyl-(2E)-3-(4-methoxyphenyl)acrylate (INCI ethylhexyl methoxy cinnamate), and isoamyl-4-methoxycinnamate (INCI isoamyl-p-methoxy cinnamate) may be named as examples for critical UV filters.

In a preferred embodiment, the sunscreen or daily care composition does not comprise Decyl b-D-glucoside (INCI decyl glucoside), 2-hydroxy-4-methoxybenzophenone (INCI oxybenzone), ethylhexyl-2-cyano-3,3-diphenyl-acrylate (INCI octocrylene), 2-ethylhexyl-(2E)-3-(4-methoxyphenyl)acrylate (INCI ethylhexyl methoxy cinnamate), and/or isoamyl-4-methoxycinnamate (INCI isoamyl-p-methoxy cinnamate).

As indicated above, the present invention relates in a second aspect to the use of an ultraviolet absorber water-dispersed composition comprising
(a) micronized 2,4,6-Tri([1,1′-biphenyl]-4-yl)-1,3,5-triazine (INCI tris-biphenyl triazine), and
(b) at least one polyglycerol monoalkyl ester
   to improve water resistance and/or the sun protection factor of a sunscreen or daily care composition comprising at least one oil soluble UVA filter, wherein the at least one oil soluble UVA filter is a combination of hexyl 2-[4-(diethylamino)-2-hydroxybenzoyl]benzoate (INCI diethylamino hydroxybenzoyl hexyl benzoate) and 1-(4-(1,1-dimethylethyl)phenyl)-3-(4-methoxyphenyl)propane-1,3-dione (INCI butyl methoxydibenzoylmethane).

It is to be understood that the improvement of the water resistance and of the sun protection factor is in respect of compositions, wherein the ultraviolet absorber water-dispersed composition is either not present or does not comprise the at least one polyglycerol monoalkyl ester but a different dispersing agent such as decyl glucoside (also known as Decyl b-D-glucoside).

The present invention is particular advantageous in view of the sun protection factor after water immersion. It is to be understood that the term “sun protection factor after water immersion” refers to the sun protection factor determined after water immersion. Hence, when the sun protection factor after water immersion of the sunscreen or daily care composition according to the present invention is improved it is improved when compared to the sun protection factor after water immersion of another sunscreen or daily care composition, wherein the ultraviolet absorber water-dispersed composition is either not present or does not comprise the at least one polyglycerol monoalkyl ester but a different dispersing agent such as decyl glucoside.

For the water resistance and the sun protection factor after water immersion several testing methods known in the art. These values are preferably determined via the solution method presented in Sohn et al., International Journal of Cosmetic Science, 2018, 1-9, since it is presently the best in vitro approach.

In a preferred embodiment, the sun protection factor after water immersion is improved.

It is to be understood that the above embodiments of the sunscreen or daily care composition are also applicable for the second aspect, in particular regarding the at least one oil soluble UVA filter, the at least one additional UV filter, and the amounts/ratios in the compositions. Further preferred embodiments of the use of the present invention to improve water resistance and/or the sun protection factor of a sunscreen or daily care composition comprising at least one oil soluble UVA filter are disclosed in the following.

According to the present invention, the polyglycerol monoalkyl ester of the use of the present invention has preferably a mean degree of polymerization of glycerol of 5 or more.

In one preferred embodiment of the use of the present invention, the at least one polyglycerol monoalkyl ester is selected from the group consisting of decaglyceryl caprate, decaglyceryl monolaurate, decaglyceryl myristate, decaglyceryl oleate, decaglyceryl stearate, decaglyceryl isostearate, hexaglyceryl cap rate, hexaglyceryl laurate, hexaglyceryl myristate, hexaglyceryl oleate, hexaglyceryl stearate, hexaglyceryl isostearate, pentaglyceryl caprate, pentaglyceryl laurate, pentaglyceryl myristate, pentaglyceryl oleate, pentaglyceryl stearate, pentaglyceryl isostearate, and combinations thereof. In a particular preferred embodiment, the at least one polyglycerol monoalkyl ester is decaglyceryl monolaurate (also known as Polyglyceryl-10 laurate).

Polyglycerol monoalkyl esters having an HLB (hydrophilic-lipophilic balance) of 14.5 or more are preferable and having an HLB of 15 or more are more preferable. The HLP value is determined by the formula

HLB=20·Mₕ/M,

wherein Mₕ is the molecular mass of the hydrophilic portion of the molecule and M is the molecular mass of the whole molecule.

Polyglycerol monoalkyl esters having an HLB of less than 14.5 may take a longer time for dispersion of micronized tris-biphenyl triazine in water phase components. Examples of polyglycerol monoalkyl esters with a mean degree of polymerization of 5 or more and having an HLB of 14.5 or more may include decaglyceryl caprate, decaglyceryl monolaurate, decaglyceryl myristate, decaglyceryl oleate, decaglyceryl stearate, decaglyceryl isostearate, hexaglyceryl laurate, pentaglyceryl laurate, pentaglyceryl myristate, pentaglyceryl stearate, and pentaglyceryl oleate, and those having an HLB of 15 or more may include decaglyceryl caprate and decaglyceryl monolaurate.

Any known process suitable for the preparation of microparticles can be used for the preparation of the micronized tris-biphenyl triazine. The sparingly soluble organic compound tris-biphenyl triazine which is used in the present invention is present in the micronized state and is preferably prepared by wet-milling processes.

As milling apparatus for the preparation of the micronized tris-biphenyl triazine there may be used, for example, a jet mill, ball mill, vibratory mill or hammer mill, preferably a high-speed mixing mill.

Tris-biphenyl triazine is micronized according to the present invention preferably in a ball mill to the desired mean particle size in the presence of water and of Polyglyceryl-10 Laurate which acts as dispersing agent. Other excipients may be added to the preparation, such as thickeners, anti-foaming agents, pH-adjusters, preservatives. Moreover, such micronized tris-biphenyl triazine formulations do not agglomerate and they remain in a dispersed form and do not readily settle.

The mean particle diameter of micronized tris-biphenyl triazine after dispersion process is preferably from 10-2000 nm, more preferably from 20-1500 nm, and further preferably ranging from 50-1000 nm. In the case of less than 10 nm, absorption in the UV-A region may be reduced, and in the case of more than 2000 nm, the ultraviolet prevention effect may be reduced and agglomeration and precipitation may be developed over time.

Micronized tris-biphenyl triazine of the use of the present invention is preferably comprised in the sunscreen or daily care composition from 0.01 to 10 wt.-%, more preferably from 0.01 to 8 wt.-%, and in particular from 0.5 to 8 wt.-%, based on the total weight of the sunscreen or daily care composition.

The at least one polyglycerol monoalkyl ester of the use of the present invention is preferably comprised in the sunscreen or daily care composition from 0.01 to 10 wt.-%, preferably from 0.01 to 8 wt.-%, and in particular from 0.1 to 5 wt.-%, based on the total weight of the sunscreen or daily care composition.

In a preferred embodiment of the use of the present invention, the ultraviolet absorber water-dispersed composition comprises from 10 to 70 wt.-%, preferably from 40 to 60 wt.-%, of micronized tris-biphenyl triazine, based on the total weight of the ultraviolet absorber water-dispersed composition.

In another preferred embodiment of the use of the present invention, the ultraviolet absorber water-dispersed composition comprises from 1 to 25 wt.-%, preferably from 2 to 20 wt.-%, of the at least one polyglycerol monoalkyl ester, based on the total weight of the ultraviolet absorber water-dispersed composition.

The weight ratio of the at least one polyglycerol monoalkyl ester to micronized tris-biphenyl triazine of the use of the present invention is preferably from 0.05 to 0.8, and more preferably from 0.1 to 0.5.

In a preferred embodiment of the use of the present invention, the ultraviolet absorber water-dispersed composition comprises from 10 to 70 wt.-%, preferably from 40 to 60 wt.-%, of micronized tris-biphenyl triazine, based on the total weight of the ultraviolet absorber water-dispersed composition and the weight ratio of the at least one polyglycerol monoalkyl ester to micronized tris-biphenyl triazine is from 0.05 to 0.8, preferably from 0.1 to 0.5.

In another preferred embodiment of the use of the present invention, the ultraviolet absorber water-dispersed composition comprises from 10 to 70 wt.-%, preferably from 40 to 60 wt.-%, of micronized tris-biphenyl triazine and from 1 to 25 wt.-%, preferably from 2 to 20 wt.-%, of the at least one polyglycerol monoalkyl ester, each based on the total weight of the ultraviolet absorber water-dispersed composition.

The ultraviolet absorber water-dispersed composition of the use of the present invention is preferably comprised in the sunscreen or daily care composition from 0.1 to 30 wt.-%, preferably from 0.5 to 25 wt.-%, more preferably from 1 to 20 wt.-%, and in particular from 2 to 16 wt.-%, based on the total weight of the sunscreen or daily care composition.

The at least one oil soluble UVA filter of the use of the present invention is preferably comprised in the sunscreen or daily care composition from 0.01 to 25 wt.-%, preferably from 0.1 to 20 wt.-%, and in particular from 2 to 15 wt.-%, based on the total weight of the sunscreen or daily care composition.

In a preferred embodiment of the use of the present invention, the sunscreen or daily care composition comprises from 0.01 to 20 wt.-%, preferably from 2 to 15 wt.-%, of the at least one oil soluble UVA filter, based on the total weight of the sunscreen or daily care composition.

In a particular embodiment of the use of the present invention, the sunscreen or daily care composition comprises from 0.01 to 10 wt.-%, more preferably from 1 to 10 wt.-%, of diethylamino hydroxybenzoyl hexyl benzoate, and from 0.01 to 5 wt.-%, more preferably from 1 to 5 wt.-%, of butyl methoxydibenzoylmethane, each based on the total weight of the sunscreen or daily care composition.

In a preferred embodiment of the use of the present invention, the sunscreen or daily care composition does not comprise Decyl b-D-glucoside (INCI decyl glucoside), 2-hydroxy-4-methoxybenzophenone (INCI oxybenzone), ethylhexyl-2-cyano-3,3-diphenyl-acrylate (INCI octocrylene), 2-ethylhexyl-(2E)-3-(4-methoxyphenyl)acrylate (INCI ethylhexyl methoxy cinnamate), and/or isoamyl-4-methoxycinnamate (INCI isoamyl-p-methoxy cinnamate).

In a preferred embodiment of the use of the present invention, the sunscreen or daily care composition further comprises at least one additional UV filter, preferably wherein the at least one additional UV filter is selected from the group consisting of water soluble UVA filters, UVB filters and/or broadband filters.

In this connection, the preferred water soluble UVA filter, UVB filters, and broadband filters are already above-mentioned.

In one preferred embodiment of the use of the present invention, the sunscreen or daily care composition comprises at least one additional UV filter selected from the group consisting of disodium phenyl dibenzimidazole tetrasulfonate, 4,4′,4″-(1,3,5-triazine-2,4,6-triyltriimino)tris-benzoic acid-tris(2-ethylhexyl)ester (INCI ethylhexyl triazone), 4,4′-[[6-[[4-[[(1,1-dimethylethyl)amino]carbonyl]phenyl]amino]-1,3,5-triazin-2,4-diyl]diimino]bis-benzoic acid-bis(2-ethylhexyl)ester (INCI Diethylhexyl Butamido Triazone), phenylbenzimidazole sulfonic acid, 2-(2H-benzotriazol-2-yl)-6-[(2-ethylhexyloxy)methyl]-4-methylphenol, bis-ethylhexyloxyphenol methoxyphenyl triazine, 2-(2H-benzotriazol-2-yl)-4-methyl-6-[2-methyl-3-[1,3,3,3-tetramethyl-1-[(trimethylsilyl)oxy]-1-disiloxanyl]propyl]phenol (INCI drometrizole trisiloxane), and combinations thereof.

In a particular embodiment of the use of the present invention, the sunscreen or daily care composition comprises 4,4′,4″-(1,3,5-triazine-2,4,6-triyltriimino)tris-benzoic acid-tris(2-ethylhexyl)ester (INCI ethylhexyl triazone) and/or bis-ethylhexyloxyphenol methoxyphenyl triazine.

In a particular embodiment of the use of the present invention, the sunscreen or daily care composition comprises 4,4′-[[6-[[4-[[(1,1-dimethylethyl)amino]carbonyl]phenyl]amino]-1,3,5-triazin-2,4-diyl]diimino]bis-benzoic acid-bis(2-ethylhexyl)ester (INCI Diethylhexyl Butamido Triazone) and/or bis-ethylhexyloxyphenol methoxyphenyl triazine.

In connection with the above embodiments, it is to be understood that the sunscreen or daily care compositions are in particularly suitable for protection of the skin from UV radiation.

Further, in connection with the above embodiments, it is to be understood that the sunscreen or daily care composition may comprise at least one additive.

In one embodiment, the at least one additive is selected from the group consisting of emulsifier, emollients, viscosity regulators (thickeners), sensory enhancers, adjuvants, preservatives, perfumes and combinations thereof.

Preferred emulsifiers include
glucose derivatives such as cetearyl glucoside, arachidyl glucoside, lauryl glucoside, polyglyceryl-3 methylglucose distearate, methyl glucose sesquistearate;
sucrose derivative such as sucrose polystearate, sucrose palmitate;
sorbitol derivatives;
glycerides of fatty acids such as glyceryl stearate, glyceryl oleate;
glumatic acid derivatives such as sodium stearoyl glutamate;
sulfosuccinic acid derivatives such as disodium cetearyl sulfosuccinate;
phosphoric acid derivatives such as potassium cetyl phosphate;
fatty acid esters of polyglyceryl such as polyglyceryl-3-diisostearate, polyglyceryl-2-dipolyhydroxystearate;
oxyalkenylated organomodified silicone/polysiloxane/polyalkyl/polyether copolymers and derivatives.

Preferred emollients include
esters of linear or branched fatty acids with linear or branched fatty alcohols such as propylheptyl caprylate, coco caprylate, isopropyl myristate, ethylhexyl palmitate;
esters of aromatic carboxylic acids with linear or branched fatty alcohols such as C₁₂-C₁₅-alkyl benzoate, ethylhexyl benzoate, phenethyl benzoate;
dicarboxylic acid esters with linear or branched alcohols such as dibutyl adipate, dicaprylyl carbonate, diisopropyl sebacate;
esters of hydroxycarboxylic acids with linear or branched fatty alcohols;
esters of linear or branched fatty acids with polyhydric alcohol such as butylene glycol dicaprylate/dicaprate;
mono-, di-, tri-glycerides based on C₆-C₁₈ fatty acids such as caprylic/capric triglycerides, coco glycerides;
guerbet alcohols such as octyldodecynol;
hydrocarbons such as hydrogenated polyisobutene, mineral oil, squalene, isohexadecane;
ethers such as dicaprylyl ether;
silicone derivatives (organomodified polysiloxanes) such as dimethylpolysiloxane, cyclic silicones.

Preferred thickeners include
fatty alcohols such as cetyl alcohol, cetearyl alcohol, stearyl alcohol;
fatty acids such as stearic acid;
fatty acid esters such as myristyl stearate;
waxes such as beeswax, carnauba wax, microcrystalline wax, ceresin, ozocerite;
polysaccharides or derivatives such as xanthan gum, guar gum, agar gum, alginates, gellan gum, carraghenan;
polyacrylates or homopolymers of reticulated acrylic acids or polyacrylamides such as carbomers, acrylate copolymers, acrylate/C₁₀-C₃₀-alkyl acrylate crosspolymer, acrylate/beheneth-25 methacrylate copolymer;
silicate derivatives such as magnesium silicates;
cellulose derivatives such as hydroxypropyl cellulose.

Preferred sensory enhancers include
polyamide derivatives such as nylon-12;
polymethyl methacrylates;
silica;
mica;
polymethylsilsesquioxane;
polyethylene;
starch derivatives such as aluminum starch octenylsuccinate;
dimethicone derivatives;
boron nitride;
HDI/trimethylol hexyllactone crosspolymer.

Preferred adjuvants include
tocopherol derivatives;
retinol derivatives;
ascorbic acid derivatives;
bisabolol;
allantoin;
panthenol;
chelating agents (EDTA, EDDS, EGTA, phytic acid, piroctone olamine);
ethylhexyl glycerin;
caprylyl glycol;
hydroxyacetophenone;
caprylhydroxymic acid;
propellants such as propane, butane, isobutene, dimethyl ether;
styrene/PVP or styrene acrylamide copolymers;
insect repellants such as butylacetylaminopropionate.

Preferred preservatives include
benzyl alcohol;
zingerone.

Preferred perfumes are selected from the group consisting of limonene, citral, linalool, alpha-isomethylionon, geraniol, citronellol, 2-isobutyl-4-hydroxy-4-methyltetrahydropyrane, 2-tert.-pentylcyclohexylacetate, 3-methyl-5-phenyl-1-pentanol, 7-acetyl-1,1,3,4,4,6-hexamethyltetraline, adipine acid diester, alpha-amylcinnamaldehyde, alpha-methylionon, amyl C butylphenylmehtylpropionalcinnamal, amylsalicylate, amylcinnamylalcohol, anisalcohol, benzoin, benzylalcohol, benzylbenzoate, benzylcinnamate, benzylsalicylate, bergamot oil, bitter orange oil, butylphenylmethylpropioal, cardamom oil, cedrol, cinnamal, cinnamylalcohol, citronnellylmethylcrotonate, lemon oil, coumarin, diethylsuccinate, ethyllinalool, eugenol, evernia furfuracea extracte, evernia prunastri extracte, farensol, guajak wood oil, hexylcinnamal, hexylcalicylate, hydroxycitronellal, lavender oil, lemon oil, linaylacetate, mandarine oil, menthyl PCA, methylheptenone, nutmeg oil, rosemary oil, sweet orange oil, terpineol, tonka bean oil, triethylcitrate, vanillin and combinations thereof.

In connection with the above preferred embodiments, it is to be understood that if the sunscreen or daily care composition comprises two or more additives, combinations of the additives as defined above are also part of the invention.

In connection with the above preferred and particularly preferred embodiments, it is to be understood that the sunscreen or daily care composition may further comprise water.

In case water is present in the sunscreen or daily care composition it is to be understood that it is preferably present in an amount of more than 5% by weight, based on the total weight of the composition. Further, it is to be understood that in case water is present in the sunscreen or daily care composition, the sunscreen or daily care composition can be an oil in water emulsion (O/W emulsion), a water in oil emulsion (W/O emulsion), a gel cream or an oil in gel.

In one embodiment of the present invention, the sunscreen or daily care composition can be provided in different forms, e.g. gels, creams, oils, lotions or in the form of a sprayable product.

The ingredients as above-outlined are in particular suitable for sensitive skin.

The present invention relates in a third aspect to the sunscreen or daily care composition as defined above for use in the administration to sensitive skin.

In one embodiment of the present invention, the sunscreen or daily care composition is applied to the skin in different forms, e.g. gels, creams, oils, lotions or in the form of a sprayable product.

In a particularly preferred embodiment of the present invention, the sunscreen or daily care composition as defined above is applied to sensitive skin. In this connection, sensitive skin is defined as skin of which the natural barrier function is weakened and has broken due to a trigger. Possible triggers are cold weather, extremely hot water, and critical components, which may be included in sunscreen or daily care compositions.

Thus, in a preferred embodiment of the present invention, a sunscreen or daily care composition as defined above, which is free of critical components such as decyl glucoside, 2-hydroxy-4-methoxybenzophenone (INCI oxybenzone), ethylhexyl-2-cyano-3,3-diphenyl-acrylate (INCI octocrylene), 2-ethylhexyl-(2E)-3-(4-methoxyphenyl)acrylate (INCI ethylhexyl methoxy cinnamate), and/or isoamyl-4-methoxycinnamate (INCI isoamyl-p-methoxy cinnamate), is applied. In this connection, it is to be understood that due to the absence of critical components such as decyl glucoside, 2-hydroxy-4-methoxybenzophenone (INCI oxybenzone), ethylhexyl-2-cyano-3,3-diphenyl-acrylate (INCI octocrylene), 2-ethylhexyl-(2E)-3-(4-methoxyphenyl)acrylate (INCI ethylhexyl methoxy cinnamate), and/or isoamyl-4-methoxycinnamate (INCI isoamyl-p-methoxy cinnamate) these compositions are especially suitable and preferred for the administration to sensitive skin.

The present invention is further illustrated by the following examples.

### Examples

The in vitro water resistance was determined via the “solution method” according to Sohn et al. “In vitro water resistance testing using SPF simulation based in spectroscopic analysis of rinsed sunscreen” Int J Cosmet Sci (2018) 1-9 using M14 EMA plates.

The M14 EMA plates are based on ethylene methacrylate acid copolymers having the dimension of 5 cm×5 cm. These conditions are understood to be the current most sufficient in vitro approach for determining the water resistance of formulations.

On each plate an amount of 2 mg/cm² (corresponding to 50 mg of formulation per plate) was applied.

Water Immersion Condition

The sunscreen-covered plates were attached using a system of strips from Tesa Hook & Loop tape at a defined height on the edges of a 1000-mL beaker (diameter of 10 cm). The beaker was filled with 500 mL of distilled water with controlled temperature of 30° C.±2° C. The water was stirred with a speed of 300 rpm for 20 min using a three-bladed propeller stirrer in a equidistant position from the attached plates. Afterwards, the plates were removed from the emptied beaker and were allowed to dry at ambient temperature for 30 min before continuation.

Solution Method

The water resistance retention was deduced from UV transmittance measurement of the respective sunscreen solution resulting from the washing of the substrate plate with and without water immersion.

In total, four plates were prepared per sunscreen. After sunscreen application followed by an equilibration period of 15 min, two of the four plates were immersed in a water bath. The two remaining plates were kept at ambient temperature in dark. After immersion, each of the four plates (two subjected and two not subjected to water immersion) was put in a separate beaker and washed using 20 mL of a solvent mixture composed of THE/ethanol/neutrol TE (50:48:2). To collect the sunscreen from the plate, each plate was rinsed 10 times using the same solvent with a Pasteur pipette holding the plate out of the solvent with tweezers to remove visually the entire sunscreen residue from the substrate plate. The solvent/formulation solution was diluted (1:40 dilution) and filled into a 1-cm quartz cuvette for UV spectroscopic measurements from 290 to 400 nm in 5 nm step using a Lambda 20 device. The result is delivered in form of absorbance data, Aᵣₛ(λ), used for the calculation of the corresponding in silico SPF value by means of a computational method developed to this purpose. The absorbance data corresponding to the two plates not subjected to water immersion delivered the static in silico SPF, whereas the data of two plates subjected to water immersion delivered the wet in silico SPF (corresponding to the value “SPF in vitro after water immersion”). As different plates are used to determine the static in silico and the wet in silico SPF, the % WRR is calculated from the ratio between the average wet and average static in silico SPF as described in the equation below:

$$\% {WRR}_{( solution method )} = \frac{( Wet in silico SPFaverage - 1 )}{( Static in si1ico SPFaverage - 1 \}} * 100$$

Further, an additional method for determining the SPF in vitro was used as follows: SPF in vitro and UVA-PF in vitro was determined based on ISO 24443, which is based on the assessment of UV-transmittance through a thin film of sunscreen sample spread on roughened substrate—SB6 PMMA-plates (4-5 μm roughness, area 4.8×4.8 cm) from Helioscreen, Paris. The sunscreen formulation to be tested was applied on the PMMA plate with an amount of 1.2 mg/cm². Three plates and 5 measurements per plate were done per formulation. The formulation was distributed manually as equal as possible on the plate. The plates applied with formulation were stored 30 minutes at room temperature and in a dark place prior the measurement.

Measurements:

Step 1

In vitro transmission measurement were performed from 290-400 nm with 1 nm steps prior to any UV irradiation with the Labsphere UV Transmittance Analyzer UV 2000S. The initial UV transmission spectrum was acquired and the SPF in vitro values were calculated before irradiation (mean SPF of all 15 values (from the three plates and 5 measurements per plate))

Step 2

A mathematical adjustment of the initial UV spectrum was performed with a coefficient “c” as described in ISO 24443 and the UVA-PF in vitro was calculated before irradiation.

Step 3

The UV irradiation with Atlas Suntest CPS+ Solar simulator of the formulation was calculated with the UV Dose D as described in ISO 24443.

Step 4

In vitro transmission measurement after UV exposure and acquisition of the UV transmission spectrum after irradiation were performed. The in vitro SPF value after irradiation was determined. (average of 15 values from the three plates and 5 measurements per plate)

Step 5

A mathematical adjustment of the UV spectrum after irradiation was performed with the same coefficient “c” (determined step 2). The UVA-PF in vitro was calculated after UV exposure.

Tris-Biphenyl Triazine dispersed with Decyl Glucoside corresponds to the product sold under the Trade Name Tinosorb A2B and comprised about 50 wt.-% of tris-biphenyl triazine and about 7.5 wt.-% of decyl glucoside.

Tris-Biphenyl Triazine dispersed with Polyglyceryl-10 laurate comprised about 50 wt.-% of tris-biphenyl triazine, about 12 wt.-% of Polyglyceryl-10 laurate, water, and other excipients such as butylene glycol and caprylyl glycol.

Formulations according to Comp. Ex. 1.1 1.2 were manufactured as follows:

Phase A and B were heated to 80° C. Phase A was added into Phase B under stirring and homogenized with an Ultra Turrax type device. The mixture was cooled down to room temperature. Phase C was added under stirring.

TABLE 1
Sunscreen formulations according to Comp. Ex. 1.1 and 1.2 and the
respective SPF in vitro after water immersion and Water resistance
in vitro

| | INCI | Comp. Ex. 1.1 | Comp. Ex. 1.2 |
|---|---|---|---|
| A | Disodium Cetearyl | 1.00 | 1.00 |
| | Sulfosuccinate | | |
| | Cetearyl Alcohol | 1.00 | 1.00 |
| | Tributyl Citrate | 4.00 | 4.00 |
| | Dibutyl Adipate | 10.00 | 10.00 |
| | Preservative | qs | qs |
| | Ethylhexyl Triazone | 0.50 | 0.50 |
| | Butyl | 5.00 | 5.00 |
| | Methoxydibenzoylmethane | | |
| B | Aqua | qsp 100% | qsp 100% |
| | Disodium EDTA | 0.20 | 0.20 |
| | Xanthan Gum | 0.40 | 0.40 |
| C | Tris-Biphenyl Triazine | 6.00* | — |
| | (TBPT) with Decyl | | |
| | Glucoside as grinding aid | | |
| | (Tinosorb A2B) | | |
| | Tris-Biphenyl Triazine | — | 6.00* |
| | (TBPT) with Polyglyceryl- | | |
| | 10 laurate as grinding aid | | |
| | SPF in vitro after water | 20 | 22 |
| | immersion | | |
| | Water resistance in vitro | 69% | 78% |

*corresponds to 3% active TBPT

Formulations according to Comp. Ex. 2 and Ex. 2 were manufactured as follows: Phase A and B were heated to 80° C. Phase A was added into Phase B under stirring and homogenized with an Ultra Turrax type device. The mixture was cooled down to room temperature. Phase C was added under stirring.

TABLE 2
Sunscreen formulations according to Comp. Ex. 2 and Ex. 2 and
the respective SPF in vitro after water immersion and Water
resistance in vitro

| | INCI | Comp. Ex. 2 | Ex. 2 |
|---|---|---|---|
| A | Disodium Cetearyl | 1.00 | 1.00 |
| | Sulfosuccinate | | |
| | Cetearyl Alcohol | 1.00 | 1.00 |
| | Tributyl Citrate | 4.00 | 4.00 |
| | Dibutyl Adipate | 10.00 | 10.00 |
| | Preservative | qs | qs |
| | Ethylhexyl Triazone | 1.00 | 1.00 |
| | Diethylamino | 2.00 | 2.00 |
| | Hydroxybenzoyl Hexyl | | |
| | Benzoate | | |
| | Butyl | 1.00 | 1.00 |
| | Methoxydibenzoylmethane | | |
| B | Aqua | qsp 100% | qsp 100% |
| | Disodium EDTA | 0.20 | 0.20 |
| | Xanthan Gum | 0.40 | 0.40 |
| C | Tris-Biphenyl Triazine | 6.00* | — |
| | (TBPT) with Decyl | | |
| | Glucoside as grinding aid | | |
| | Tris-Biphenyl Triazine | — | 6.00* |
| | (TBPT) with Polyglyceryl- | | |
| | 10 laurate as grinding aid | | |
| | SPF in vitro after water | 15 | 16 |
| | immersion | | |
| | Water resistance in vitro | 60% | 75% |

*corresponds to 3% active TBPT

Formulations according to Comp. Ex. 3.1 and 3.2 and according to Ex. 3 were manufactured as follows:

Phase A and B were heated to 80° C. Phase A was added into Phase B under stirring and homogenized with an Ultra Turrax type device. The mixture was cooled down to room temperature. Phase C was added under stirring.

TABLE 3
Sunscreen formulations according to Comp. Ex. 3.1 and 3.2 and Ex. 3 and the
respective SPF in vitro after water immersion and Water resistance in vitro

| | INCI | Comp. Ex. 3.1 | Comp. Ex. 3.2 | Ex. 3 |
|---|---|---|---|---|
| A | Disodium Cetearyl | 1.00 | 1.00 | 1.00 |
| | Sulfosuccinate | | | |
| | Cetearyl Alcohol | 1.00 | 1.00 | 1.00 |
| | Tributyl Citrate | 4.00 | 4.00 | 4.00 |
| | Dibutyl Adipate | 10.00 | 10.00 | 10.00 |
| | Preservative | qs | qs | qs |
| | Ethylhexyl Triazone | 1.00 | 1.00 | 1.00 |
| | Diethylamino | 3.00 | — | 2.00 |
| | Hydroxybenzoyl Hexyl | | | |
| | Benzoate | | | |
| | Butyl | — | 3.00 | 1.00 |
| | Methoxydibenzoylmethane | | | |
| B | Aqua | qsp 100% | qsp 100% | qsp 100% |
| | Disodium EDTA | 0.20 | 0.20 | 0.20 |
| | Xanthan Gum | 0.40 | 0.40 | 0.40 |
| C | Tris-Biphenyl Triazine | 6.00* | 6.00* | 6.00* |
| | (TBPT) with Polyglyceryl- | | | |
| | 10 laurate as grinding aid | | | |
| | SPF in vitro | 21 | 19 | 24 |
| | SPF in vitro after water | 14 | 13 | 16 |
| | immersion (solution | | | |
| | method) | | | |
| | Water resistance in vitro | 67% | 59% | 75% |

*corresponds to 3% active TBPT

Surprisingly, best water resistance (in vitro) and SPF (in vitro) values are being obtained with Ex. 3.

1.-13. (canceled)

14. A sunscreen or daily care composition comprising
(i) an ultraviolet absorber water-dispersed composition comprising
(a) micronized 2,4,6-Tri([1,1′-biphenyl]-4-yl)-1,3,5-triazine (INCI tris-biphenyl triazine), and
(b) decaglyceryl monolaurate (INCI Polyglyceryl-10 Laurate); and
(ii) at least one oil soluble UVA filter,
wherein the at least one oil soluble UVA filter is a combination of hexyl 2-[4-(diethylamino)-2-hydroxybenzoyl]benzoate (INCI diethylamino hydroxybenzoyl hexyl benzoate) and 1-(4-(1,1-dimethylethyl)phenyl)-3-(4-methoxyphenyl)propane-1,3-dione (INCI butyl methoxydibenzoylmethane).

15. The sunscreen or daily care composition according to claim 14, further comprising at least one additional UV filter.

16. The sunscreen or daily care composition according to claim 14, further comprising at least one additional UV filter selected from the group consisting of water soluble UVA filters, UVB filters, broadband filters, and combinations thereof.

17. The sunscreen or daily care composition according to claim 14, comprising
(i) from 0.01 to 10 wt.-% of micronized 2,4,6-Tri([1,1′-biphenyl]-4-yl)-1,3,5-triazine (INCI tris-biphenyl triazine), and
from 0.01 to 8 wt.-% of decaglyceryl monolaurate (INCI Polyglyceryl-10 Laurate); and
(ii) from 0.01 to 20 wt.-% of the at least one oil soluble UVA filter,
each based on the total weight of the sunscreen or daily care composition.

18. The sunscreen or daily care composition according to claim 14, comprising
(i) from 0.01 to 10 wt.-% of micronized 2,4,6-Tri([1,1′-biphenyl]-4-yl)-1,3,5-triazine (INCI tris-biphenyl triazine), based on the total weight of the sunscreen or daily care composition, and
wherein the weight ratio of decaglyceryl monolaurate (INCI Polyglyceryl-10 Laurate) to micronized 2,4,6-Tri([1,1′-biphenyl]-4-yl)-1,3,5-triazine (INCI tris-biphenyl triazine) is from 0.05 to 0.8 and
(ii) from 0.01 to 10 wt.-% of diethylamino hydroxybenzoyl hexyl benzoate, based on the total weight of the sunscreen or daily care composition, and from 0.01 to 5 wt.-% of butyl methoxydibenzoylmethane, based on the total weight of the sunscreen or daily care composition.

19. The sunscreen or daily care composition according to claim 14, wherein the sunscreen or daily care composition does not comprise Decyl b-D-glucoside (INCI decyl glucoside), 2-hydroxy-4-methoxybenzophenone (INCI oxybenzone), ethylhexyl-2-cyano-3,3-diphenyl-acrylate (INCI octocrylene), 2-ethylhexyl-(2E)-3-(4-methoxyphenyl)acrylate (INCI ethylhexyl methoxy cinnamate), or isoamyl-4-methoxycinnamate (INCI isoamyl-p-methoxy cinnamate).

20. A method for improving water resistance and/or the sun protection factor of a sunscreen composition or daily care composition comprising at least one oil soluble UVA filter, wherein the at least one oil soluble UVA filter is a combination of hexyl 2-[4-(diethylamino)-2-hydroxybenzoyl]benzoate (INCI diethylamino hydroxybenzoyl hexyl benzoate) and 1-(4-(1,1-dimethylethyl)phenyl)-3-(4-methoxyphenyl)propane-1,3-dione (INCI butyl methoxydibenzoylmethane), the method comprising:
(a) micronized 2,4,6-Tri([1,1′-biphenyl]-4-yl)-1,3,5-triazine (INCI tris-biphenyl triazine), and
(b) at least one polyglycerol monoalkyl ester.

21. The method according to claim 20, wherein the at least one polyglycerol monoalkyl ester is selected from the group consisting of decaglyceryl caprate, decaglyceryl monolaurate, decaglyceryl myristate, decaglyceryl oleate, decaglyceryl stearate, decaglyceryl isostearate, hexaglyceryl cap rate, hexaglyceryl Iaurate, hexaglyceryl myristate, hexaglyceryl oleate, hexaglyceryl stearate, hexaglyceryl isostearate, pentaglyceryl caprate, pentaglyceryl Iaurate, pentaglyceryl myristate, pentaglyceryl oleate, pentaglyceryl stearate, pentaglyceryl isostearate, and combinations thereof.

22. The method according to claim 20, wherein the ultraviolet absorber water-dispersed composition comprises
from 10 to 70 wt.-% of micronized 2,4,6-Tri([1,1′-biphenyl]-4-yl)-1,3,5-triazine (INCI tris-biphenyl triazine), and/or
from 1 to 25 wt.-% of the at least one polyglycerol monoalkyl ester, each based on the total weight of the ultraviolet absorber water-dispersed composition.

23. The method according to claim 20, wherein the sunscreen or daily care composition comprises
from 0.01 to 20 wt.-% of the at least one oil soluble UVA filter, based on the total weight of the sunscreen or daily care composition.

24. The method according to claim 20, wherein the sunscreen or daily care composition does not comprise Decyl b-D-glucoside (INCI decyl glucoside), 2-hydroxy-4-methoxybenzophenone (INCI oxybenzone), ethylhexyl-2-cyano-3,3-diphenyl-acrylate (INCI octocrylene), 2-ethylhexyl-(2E)-3-(4-methoxyphenyl)acrylate (INCI ethylhexyl methoxy cinnamate), and/or isoamyl-4-methoxycinnamate (INCI isoamyl-p-methoxy cinnamate).

25. The method according to claim 20, wherein the sunscreen or daily care composition further comprises at least one additional UV filter.

26. A method for administering a composition to sensitive skin comprising the sunscreen or daily care composition as defined in claim 14.
